(19) United States
(12) Patent Application Publication
(10) Pub. No.: US 2009/0326582 A1
(43) Pub. Date: Dec. 31, 2009

(54) Dynamic Rod

(76) Inventors: Marcus Songer, Marquette, MI (US); Jason Songer, Munster, IN (US)

Correspondence Address: BRIAN JANOWSKI, 375 RIVER PARK CIRCLE, MARQUETTE, MI, 49855, US

(21) Appl. No.: 12/422,044

(22) Filed: Apr. 10, 2009

Related U.S. Application Data
(60) Provisional application No. 61/043,869, filed on Apr. 10, 2008.

(51) Int. Cl. A61B 17/70; A61B 17/86

(52) U.S. Cl. 606/255; 606/264; 606/305

(57) ABSTRACT

A spinal implant system for stabilization of the spine is disclosed comprising a pair of bone anchors, an elongate stabilization device received in the bone anchors, the stabilization device having an elongate inner stabilizing member and an outer stabilizing member disposed about the inner member and wherein said anchors are configured to inhibit translation of the outer member and to permit translation of the inner member.

### CROSS-REFERENCE TO RELATED APPLICATIONS

The subject application is a utility application stemming from U.S. provisional application Ser. No. 61/043,880 filed Apr. 10, 2008 the disclosure of which is herein incorporated by reference in its entirety.

### FIELD OF THE INVENTION

The spinal stabilization implant system disclosed herein is designed to provide a predetermined stabilization constraint to the natural spine within beneficial motion and flexibility limits.

### BACKGROUND OF THE INVENTION

A human spine comprises a number of joints often referred to motion segments. These segments exhibit kinematics characteristic of the entire spine. The motion segments are capable of flexion, extension, lateral bending and translation. The components of each motion segment are important for the stability of the joint and each unit include two adjacent vertebrae and their apophyseal joints, the intervertebral disc, and the connecting ligamentous tissue.

Components of a motion segment that move out of position or become damaged can lead to serious pain and may lead to further injury to other components of the spine. Depending upon the severity of the structural changes that occur, treatment may include fusion, discectomy, and laminectomy.

Underlying causes of structural changes in the motion segment unit leading to instability include trauma, degeneration, aging, disease, surgery, and the like. Thus, rigid stabilization of the motion segment unit may be the most important element of a surgical procedure in certain cases (i.e., injuries, deformities, tumors, etc.), whereas it is a complementary element in others (i.e., fusion performed due to degeneration). The purpose of rigid stabilization is the immobilization of a motion segment unit.

The rigid design of systems common in the prior art typically cause stress concentrations and contribute to the degeneration of the joints above and below the fusion site. In addition, rigid, bar-like elements eliminate the function of the motion segment unit.

Fusion procedures can be improved by modifying the load sharing characteristics of the treated spine. A need exists in the art for a soft spine stabilization system that replicates the physiologic response of a healthy motion segment.

### SUMMARY OF THE INVENTION

This disclosure encompasses stabilization systems for spinal motion segments. In particular, the present invention is directed to various embodiments of a soft stabilization system comprising a specialized elongated fixation member having an outer elongated member surrounding an inner elongated member. The system further comprises at least two specialized bone anchors designed typically in the form of pedicle screws to restrain the outer elongated member without compressing the inner elongated member thereby causing undesired wear of components.

The system described herein has many benefits over earlier soft fixation systems. This system can easily span multiple vertebral levels since multiple pedicle screws can be attached to one elongated fixation member thereby providing multi-level soft stabilization even during a minimally invasive surgery. Competitive systems by their design do not allow multiple level soft fixation. The elongated member in this system can be contoured or bent anywhere along the rod whereas other soft stabilization systems have limited or no ability to create an even bend unless it is built into the system initially. There are no stress concentrations on the elongated fixation member since this member is a combination of continuous materials vs. the multiple components of rods in the prior art which are assembled and have combinations of stiff and elastic combinations along the rod.

Other benefits include: consistent stiffness along the length of the elongated fixation member thereby providing flexibility in fixing screws anywhere along this member with no required distance between the screws. Also, various outer member sleeve sizes can accommodate to various sizes of yolks making it potentially compatible with many different pedicle screw systems. Further, the elongated fixation member can be inserted in a minimally invasive fashion—pericutaneously. All other systems have to be inserted into the yolk of a pedicle screw at specific points, usually under direct vision. Since the rod is made of the combination of the same materials continuously along its length, it can be blindly inserted into a yolk of a pedicle screw. Additionally the stiffness of this soft fixation system can be adjusted to the relative size, weight and functional demands of the patient by selecting different inner stabilization member materials and elastic outer stabilization member materials.

Additional benefits include the system would be the only one that could be assembled intra-operatively based on testing of the patients relative flexibility or stiffness measured intra-operatively. The diameter of the elongated fixation member would not be needed to be changed to increase or decrease stiffness which currently is required of systems in the prior art. Stated otherwise, the prior art systems attempt to vary the size or length of elastic and rigid components to increase or decrease stiffness. The system disclosed herein is capable of easy exchange of components of various materials or the relative thicknesses of the inner rigid member and outer elastic components. The system can be pre-assembled by the manufacturer or assembled by the surgeon to meet specific physical demands of a patient or other surgical goals. A family of products that vary in both ability to bend in the saggital and coronal planes, as well as an ability to elongate with flexion and extension is contemplated.

Finally, this dynamic rod concept has less risk of fatigue fracture due to the uniformity along the rod and lack of stress risers which have plagued other systems.

### BRIEF DESCRIPTION OF THE DRAWINGS

FIG. 1 is a partially exploded view of a preferred embodiment of the spinal implant system.

FIG. 2 is a perspective view of the spinal implant system.

FIG. 3 is a partially exploded view of a preferred embodiment of the elongated stabilization assembly.

FIG. 3A is an alternative perspective view of an outer elongated member in the form of a coiled spring.

FIG. 3B is an alternative perspective view of an inner elongated member.

FIG. 3C is an alternative perspective view of the lead tip.

FIG. 4 is a perspective view of a preferred embodiment of the elongated stabilization assembly.

FIG. 5 is a perspective view of an alternative outer elongated member having a ribbed outer surface.

FIG. 6 is a section view of the ribbed alternative outer elongated member shown in FIG. 5.

FIG. 7 is a detail view of the ribbed alternative outer elongated member shown in FIG. 5.

FIG. 8 is a perspective view of a second alternative outer elongated member.

FIG. 9 is a section view of the alternative outer elongated member shown in FIG. 8.

FIG. 10 is a perspective view of a third alternative outer elongated member.

FIG. 11 is a detail view of recesses in the surface and wall of the outer elongated member illustrated in FIG. 10.

FIG. 12 is a section view of the third alternative outer elongated member shown in FIG. 10.

FIGS. 13A & 13B is a perspective and detailed view of the outer elongated member in FIG. 10 with the addition of a grooved outer surface.

FIGS. 14A & 14B is a perspective and detailed view of an embodiment of an outer elongated member having a shaped or knurled outer surface portion.

FIG. 15 is a perspective view of a polyaxial pedicle screw having a threaded cap and configured to restrain the elongated stabilization assemblies shown in FIG. 4 and elsewhere.

FIG. 16 is an exploded perspective view of a polyaxial pedicle screw having a threaded cap and configured to restrain the elongated stabilization assemblies shown in FIG. 4 and elsewhere.

FIG. 17A-D illustrates various views of a threaded pedicle screw cap assembly configured to restrain the elongated stabilization assemblies shown in FIG. 4 and elsewhere.

FIG. 18A-C illustrates various views of the upper cap portion of the threaded pedicle screw cap assembly illustrated in FIG. 17.

FIG. 19A-E illustrates various views of the upper saddle portion of the threaded pedicle screw cap assembly illustrated in FIG. 17.

FIG. 20A-C illustrates various views of the polyaxial pedicle screw portion illustrated FIG. 16 and elsewhere.

FIG. 21A-D illustrates various views of the lower saddle portion of the polyaxial pedicle screw illustrated in FIG. 16 and elsewhere.

FIG. 22A-D illustrates various views of the polyaxial pedicle screw yoke shown in FIG. 16 and elsewhere.

FIG. 23A-B illustrates an exploded and assembled perspective view of a fixed pedicle screw with a lower saddle machined into the yoke.

FIG. 24A-B illustrates an exploded and assembled perspective view of a fixed pedicle screw comprising a removable lower saddle.

FIG. 25A-C illustrates various views of a lower saddle for a fixed pedicle screw configured to restrain an elongated stabilization assembly as disclosed herein.

FIG. 26A-B illustrates an exploded and assembled perspective view of a polyaxial pedicle screw utilizing a non-threaded insertion cap.

FIG. 27A-B illustrates an exploded and assembled perspective view of a non-threaded insertion cap configured to restrain the elongated stabilization assemblies shown in FIG. 4 and elsewhere.

FIG. 28A-D illustrates various views of the upper cap portion of the non-threaded pedicle screw cap assembly illustrated in FIG. 26.

FIG. 29A-C illustrates various views of the polyaxial pedicle screw yoke shown in FIG. 26.

### DETAILED DESCRIPTION

This disclosure describes a spinal stabilization system comprising a specialized elongated fixation member and at least two specialized bone fasteners designed to restrain the elongated fixation member thereby softly stabilizing the associated spinal segments. The elongated fixation member comprises an outer elongated member surrounding an inner elongated member. The specialized bone fasteners/anchors restrain the outer elongated member without substantial compression on the inner member and without inhibiting translatory motion of the inner elongated member with respect to the outer member.

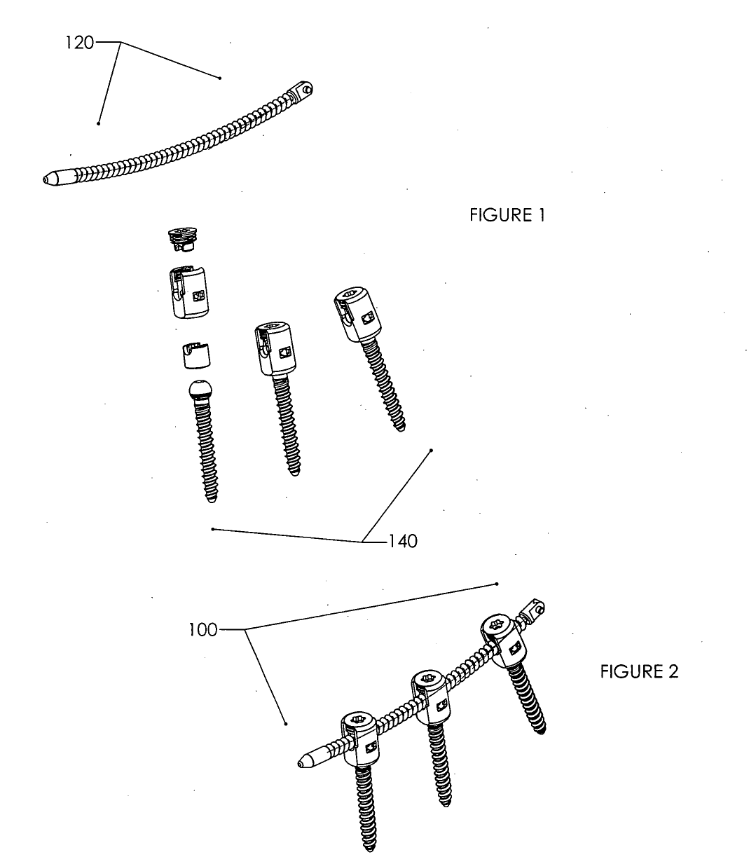

FIG. 1 illustrates a preferred embodiment of the spinal stabilization system 100 disclosed herein in a partially exploded form. The system 100 comprises an elongated stabilization member 120 and at least two fixed or polyaxial bone anchors in the form of pedicle screws 140 configured to restrain the elongated stabilization member 120. This embodiment of the fully assembled system illustrated in FIG. 2 as would be implanted in a spine spanning 2 vertebral levels. The overall length of the stabilization member can be adjusted and more screws 140 can be utilized to span a single vertebral level or multiple levels.

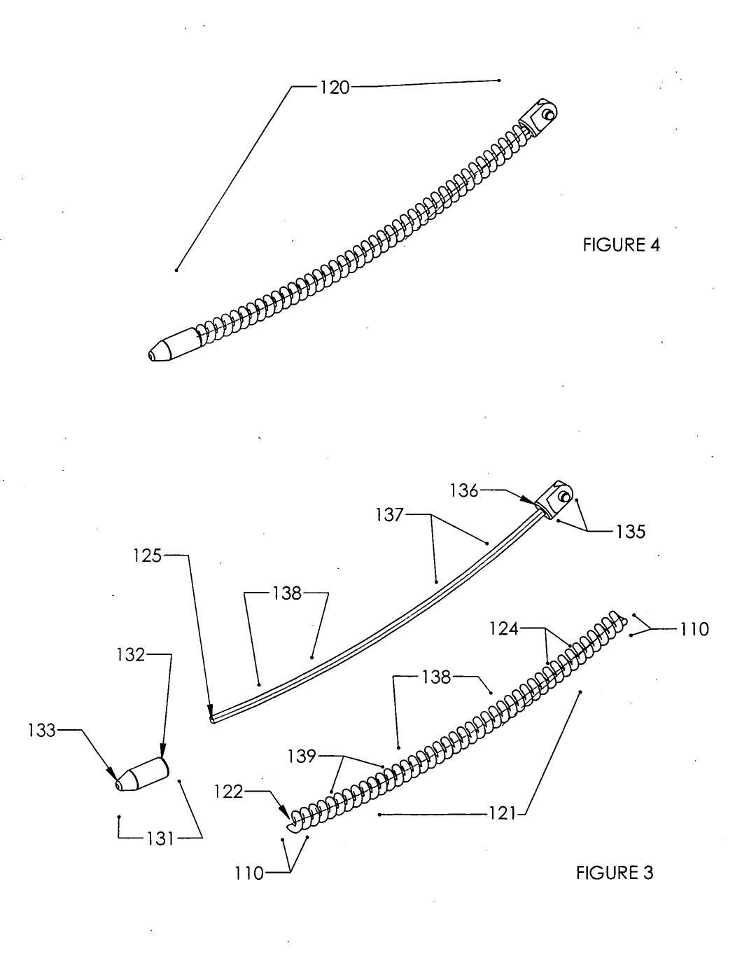

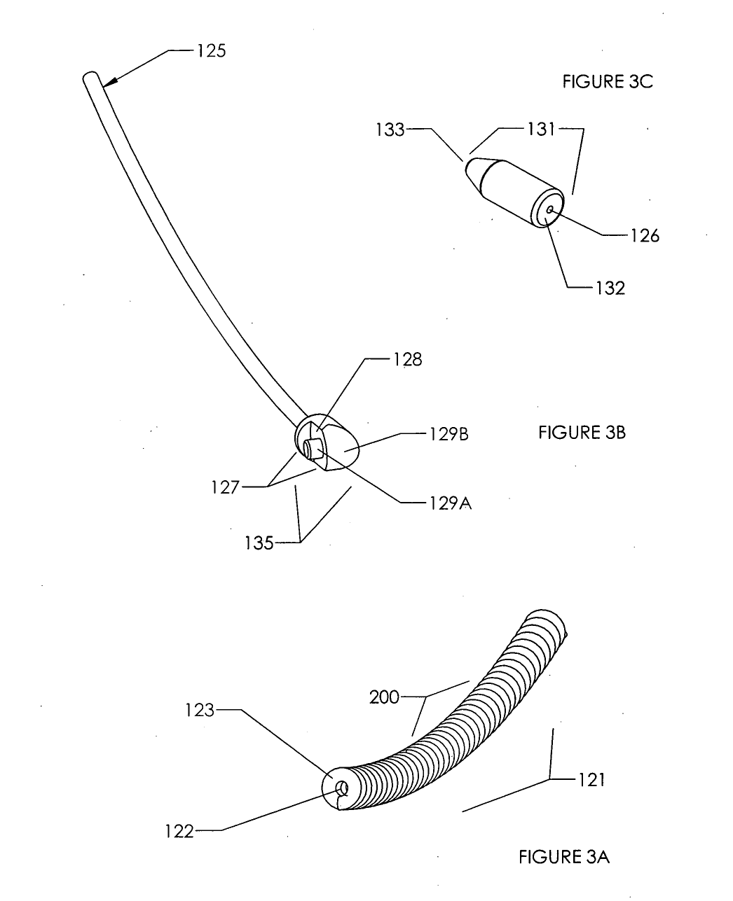

As seen in FIGS. 3 and 4, the elongated stabilization member 120 comprises an elastic elongated outer member 121 represented as a coiled spring in this embodiment. Member 120 also comprises an elongated inner member 130, preferably in the form of a solid rod to control the bendability of the construct. The elastic outer member controls the torsion and elongation of the elongated stabilization member construct.

Inner member 130 is preferably made from carbon fiber, PEEK or similar polymers, titanium, or titanium alloys, cobalt chrome, stainless steels, but may also be manufactured from other biocompatible materials. The inner member 130 comprises an inner member surface portion 137 which may have a low wear coating 138 to improve wear and decrease friction between the inner member surface portion 137 and the outer member 121 as the two members 121 and 130 move with respect to each other. It is preferred that the inner member 130 has a circular cross section, although not required, and is smooth across its surface to further ease movement of the outer member 121 across the inner member surface 137.

Similarly, the outer member also comprises an outer member surface portion 139. Alternatively, surface portion 139 may have a low wear coating 138. Depending on the materials chosen for each member 121, 130, the surface portions may not require a low wear coating, have only one of the surfaces 137, 139 coated, or both surfaces may be coated. For example, the inner member 130 may be manufactured from cobalt chrome and coated in PEEK while the outer member 121 is manufactured from nitinol. Alternatively as example, the inner member 130 may be manufactured of PEEK and coated with titanium or cobalt chrome.

The inside cannulation profile 122 of outer member 121 preferably matches the outside profile of the inner member 130 with adequate gapping between the surfaces 137 139 for smooth gliding movement therebetween. Although the inner member 130 embodiment shown in FIG. 3 has a preferred circular cross section, it is recognized that the cross section could be oval or other non-circular shape provided the outer member 121 and screws 140 are adapted to accommodate the non-circular profile.

The outer member 121 functions as a flexible elastic housing preferably in the form of a tube, a cannulated rod, or spring. As seen in the preferred embodiment in FIG. 3A, the outer member 121 is in the form of a coiled spring wherein the round spring coils 123 form a circular cannulation through the center of the outer member 121. The spring may comprise compression gaps 124 which will provide for a gradual increased spring resistance between the screws 140 as the spring undergoes compression due to spinal extension forces exerted by the screws 140. Once the screws 140 move in relation to a predetermined amount of spinal extension, these compression gaps 124 will close to prevent further spinal extension.

Similarly in spinal flexion, the screws 140 will move apart and the outer member 121 will become stiffer as the member 121 is extended past its neutral point. As the screws 140 approximate the lead tip 131 and the instrument tip 135, the screws 140 and thus spinal flexion will eventually be stopped as the outer member 121 compresses against stops 132 and 136. If the compression gaps 124 directly adjacent stops 132 and 136 are closed, the spine will be prevented from further flexion. Also limiting flexion is the portion of the spring situated between the screws 140. During spinal flexion, this portion of the spring is pulled into spring extension and become stiffer thereby also assisting in limiting flexion motion.

The above paragraphs describe the outer member 121 bias action for a stabilization system 100 applied to a spine in a neutral position. However, components of this system 100 have several means for creating a variety of affects. For example, if the system 100 is implanted in the neutral spine with the outer member 121 intermediate the screws 140 in slight compression, the system 100 may be used to open the gaps between the vertebral bodies and relieve compression and pain that may be exerted on nerves exiting the spinal canal.

There are a multitude of other adjustments that can be made to the elongated stabilization member 120. For example, material choices for the outer member 121 and for the inner member 130 will greatly influence the stiffness of the member 120. As will be described later, the stabilization member 120 may be assembled according to the surgeon's specifications inside or outside the operating room. Therefore it is foreseen that the surgeon may make choices for an inner member 130 such as diameter, material stiffness, and overall length. Likewise the surgeon may also make choices for an outer member 121 such as coils/inch, material stiffness, coil inner/outer diameter, spring constant, inner/outer member length ratio, etc. The variety of choices for each of these variables will provide the skilled surgeon ample opportunity to adjust the elongated stabilization member 120. The system 100 is therefore adaptable to a spectrum of patients of various sizes, shapes, weights, and spinal conditions. In this manner the system 100 may come in the form of a kit with a variety of parts to be assembled to the surgeon's preference. As such the lead tip 131 and/or the instrument tip 135 may be removable from the inner member 130 for mounting various outer members 121 therebetween. If only one tip 131 135 is removable, the other may be integral to the manufacture of the inner member 130. Otherwise, the tips may be restrained to the inner member 130 by common connections such as machine threads, bayonet connection, welding, pinning, mohr's taper, press fit, chemical bonding, or other similar fastening mechanisms. FIG. 3 illustrates the lead end of the inner member 130 having an inner member connection portion 125 in the form of threads in this embodiment. Complimenting this is a tip connection portion 126 also in the form of threads in this embodiment.

For convenience sake, the elongated stabilization member 120 may come preassembled wherein the surgeon only has to choose a preassembled member 120 meeting his or her predetermined requirements. The elongated stabilization member 120 may also come pre-bent, as seen in FIGS. 3 and 4 typically to match the natural curvature of the neutral spine. However the stabilization member 120 may be manufactured straight. In either case, the surgeon has the option of bending the stabilization member 120 with a bender specially designed for this purpose and further designed not to damage the outer member 121.

The instrument tip 135 comprises structure for connection to a rod inserter instrument. As seen in FIG. 4, it is preferable if the instrument tip 135 and the lead tip 131 are generally no larger than the diameter of the outer member 121. This streamlined profile of the elongated stabilization member 120 is a particular benefit when used in a minimally invasive surgery as the member 120 can be passed down a tube through the tissues of the skin, fascia, and muscle and into the screws 140 for final fixation. Since the outer member 121 extends substantially the length of the inner member 130, the stabilization member 120 typically does not require precise visual placement within the screws 140 which ultimately means less surgical incision is required. The instrument tip 135 preferably comprises an instrument connector portion 127. In this embodiment, the instrument connector portion 127 comprises a face portion 128 and a mounting pin or recess 129A for grasping by an inserter instrument. A contemplated inserter for this service comprises a complementary face on the instrument to mate with the face portion 128, as well as a complimentary pin or recess to mate with pin or recess 129A. Once the elongated inserter instrument is mounted to the complimentary structure, a sleeve is slid down the shaft of the instrument over the outer instrument tip body 129B to securely hold the elongated stabilization member 120 to the instrument.

The lead tip 131 has a nose 133 configured for entry through the soft tissues normally encountered in a spine surgery. A particular benefit of this spinal system is that it is configured for use minimally invasively if so desired wherein the nose 133 may be shaped to have a bullet shaped tip for easy movement through tissue. In addition, the pedicle screws 140 of this system may be fixed on infinite points of the outer member 121 thereby requiring far less invasive viewing for precise placement of the elongated stabilization member 120 compared to soft fixation systems of competitors.

The lead tip 131 and the instrument tip 135 are configured as generally flat stops against the ends of the outer member 121. Unlike that shown in FIGS. 3 and 3A, the ends 110 of outer member 121 are preferably finished to be flatted to create a low wear interface between the ends 110 and the stops 132 and 136. In addition, a low wear polymer washer or coating may be utilized. Although flattened ends 110 and stops 132 and 136 are preferred, it is apparent that other non-flattened interfaces will also work well as long as they provide for a low stress low wear interface.

For increased torsion resistance, tips 131 and 135 may be modified to include restraining structure (i.e. clamping bands, set screws, pinning) to restrain one or more ends of the outer member 121 thereby minimizing rotational or torsional movement of the outer member 121 about the inner member 130.

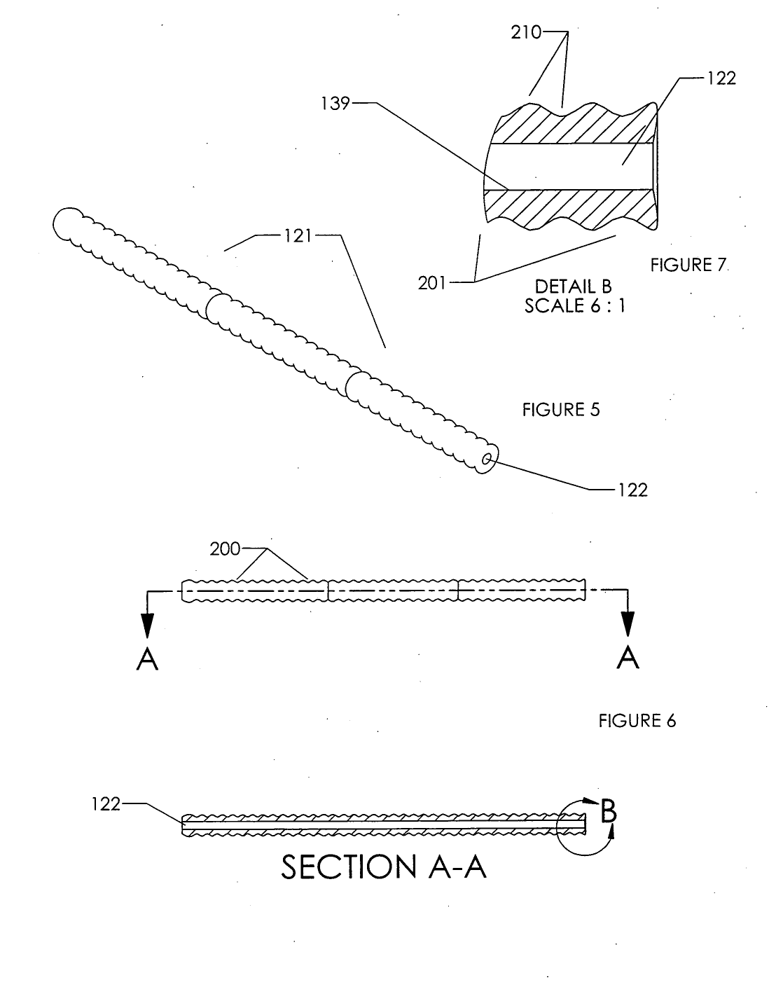

Alternative embodiments of the outer member 121 are illustrated in FIGS. 5-14. The embodiments are preferably manufactured in the form of an elastomeric polymer such as a polyurethane or similar material. Certain biocompatible metals such as nitinol with elastomeric properties may also be appropriate. In each of these embodiments, the outer member 121 comprises an outer member surface portion 139. The inside cannulation profile 122 of outer member 121 preferably matches the outside profile of the inner member 130 with adequate gapping between the adjacent surfaces 137 & 139 for smooth gliding low wear movement therebetween. These outer member embodiments are absent the coiled structure illustrated in FIG. 3A as they tend to rely on the greater elastomeric properties of the material to provide similar functional benefits.

The outer member 121 embodiment illustrated in FIGS. 5-7 comprises an outer surface portion 200 configured for restraint by a screw 140. In this embodiment, the outer surface portion 200 is configured with a restraint surface structure 201 in the form of radial ribs or grooves 210 complementing the screw 140 restraint structure to be described later. The restraint surface structure 201 provides a physical engagement structure, as opposed to a smooth level surface, for secure restraint by screws 140. The ribs 210 are configured to a predetermined depth so to not significantly weaken the wall of the outer member 121.

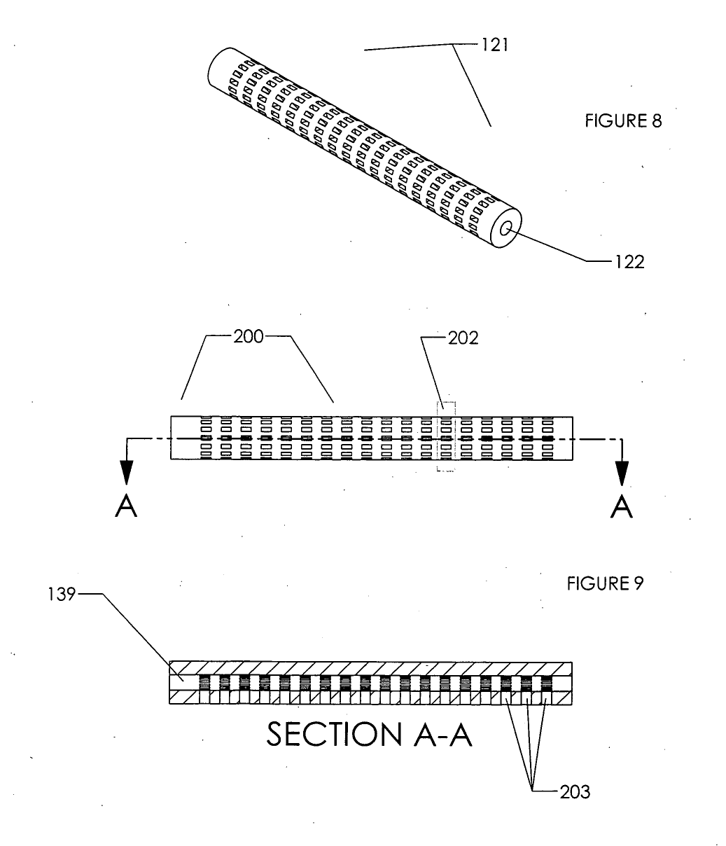

The outer member 121 embodiment illustrated in FIGS. 8 & 9 comprises an outer surface portion 200 configured for restraint by a screw 140. In this embodiment, the outer surface portion 200 is configured with restraint wall structure 202 complementing the screw 140 restraint structure to be described later. This restraint wall structure 202, implemented here in the form of recesses 203, provide a physical engagement structure, as opposed to a smooth level surface, for secure restraint by screws 140. The recesses 203 are configured to a predetermined depth so to not significantly weaken the wall of the outer member 121. In this embodiment the recesses 203 are in the form of a rectangle extending through the wall of outer member 121. Alternatively, the recesses 202 may extend only partially through the outer member 121 to a predeterminded depth suitable for adequate restraint engagement by the screws 140.

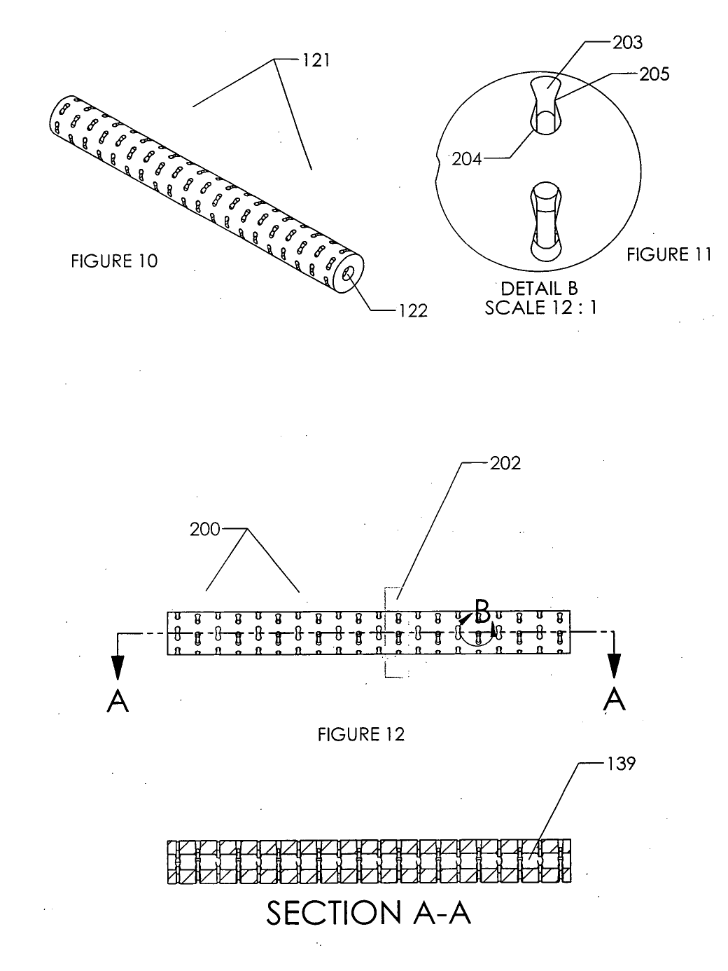

A preferred implementation of the restraint wall structure 202 is illustrated in the embodiment of FIG. 10-12. In this embodiment the recesses 203 in the outer member 121 have the shape similar to the number 8 in a radial pattern about the surface of the outer member 121. Unlike recesses 203 in FIG. 8, the recesses 203 in FIG. 11 have radiused corners 204 thereby reducing stress concentrations at these points and reducing the likelihood of outer member 121 material failure. In addition, each row of the radial 8 shaped recesses 203 are offset thereby dispersing stress more evenly through the material. In addition, the number 8 profile is preferred over a simple oval profile since the 8 profile will better tolerate stresses due to extension of the outer member 121 as well as serving as bumper stops 205 if outer member undergoes compression.

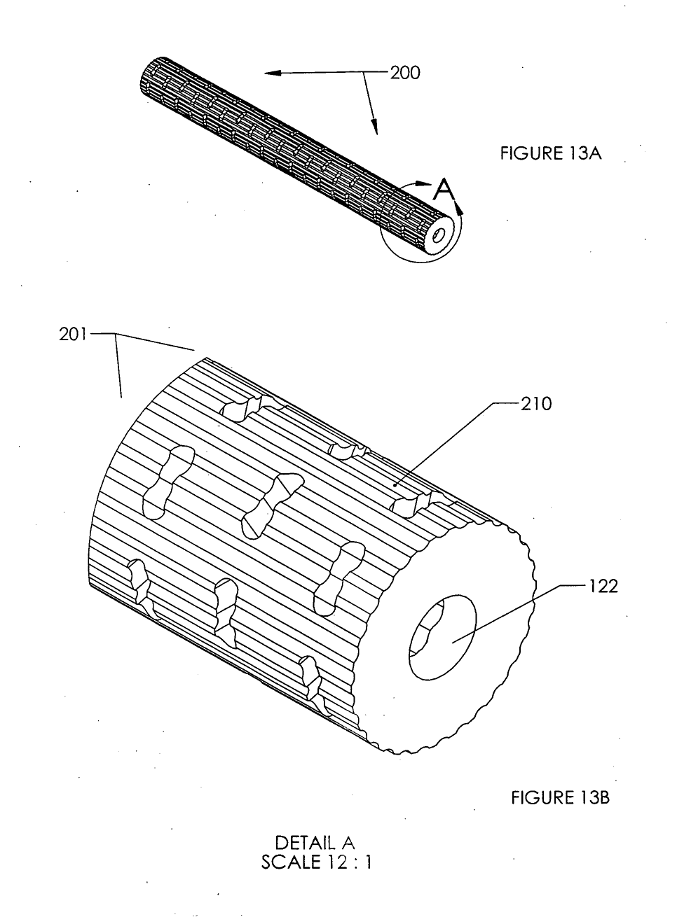

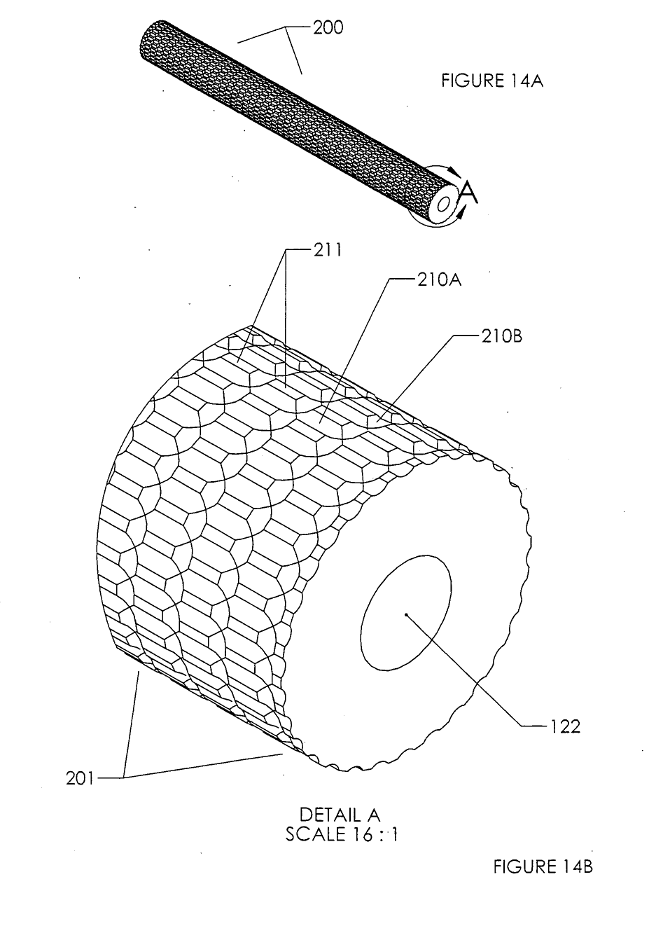

The outer member 121 embodiment illustrated in FIGS. 13A and 13B is similar to the embodiment in FIG. 10 except that outer surface portion 200 also comprises restraint surface structure 201 implemented as a series of longitudinal ribs in this embodiment. This restraint surface structure 201 provides a physical engagement structure, as opposed to a smooth level surface, for secure restraint by screws 140. The recesses 203 are configured to a predetermined depth so to not significantly weaken the wall of the outer member 121.

In yet another example, FIGS. 14A & 14B illustrate an outer member 121 having a shaped or knurled outer surface portion 200. The patterns may be varied. In this embodiment ribs or grooves 210A are formed in a longitudinal pattern with crossing ribs or grooves 210B formed in a radial pattern. This pattern creates a multitude of surface bosses 211 which together create a restraint surface structure 201 providing a physical engagement structure, as opposed to a smooth level surface, for secure restraint by screws 140. Recesses 203, such as those illustrated in FIG. 11, may be added if so desired for further restraint or to vary the overall stiffness or extendability of the outer member 121.

In a final example, an outer member 121 manufactured from a polymer may include an integral metallic spring member, preferably coiled, (not shown) molded within the polymer. This integral spring member may add beneficial spring characteristics that a polymer outer member 121 could not achieve alone.

Now described in detail are several embodiments of fixed and variable angle pedicle screws illustrating modifications to make them suited to restrain the outer member 121 of the elongated stabilization member 120 thereby creating a functioning spinal stabilization system 100 as disclosed herein.

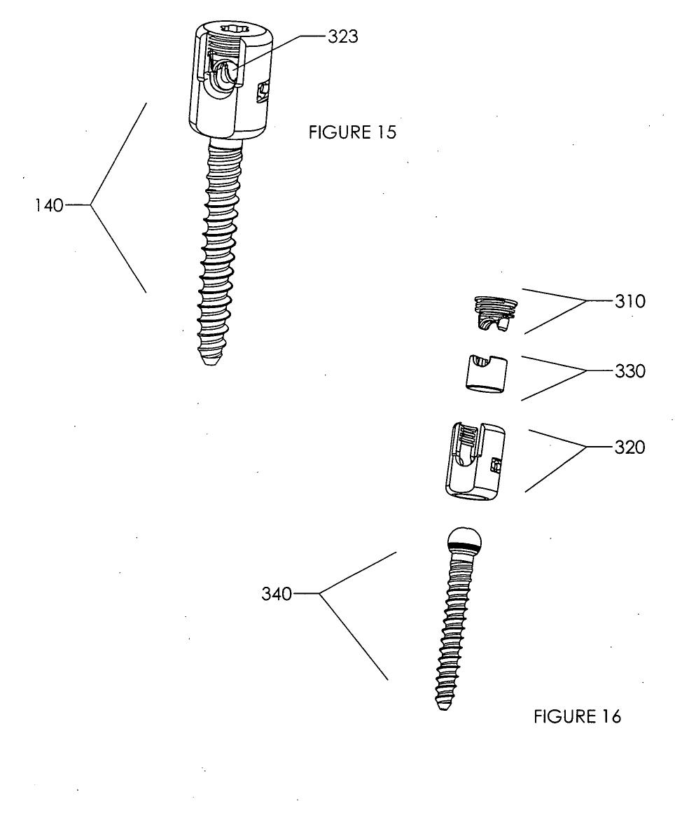

In the preferred embodiment, a pedicle screw 140 of the threaded poly-axial variety is illustrated in FIG. 15. This screw comprises a locking cap assembly 310, a poly-axial yoke 320, a lower saddle 330, and a poly-axial bone screw 340.

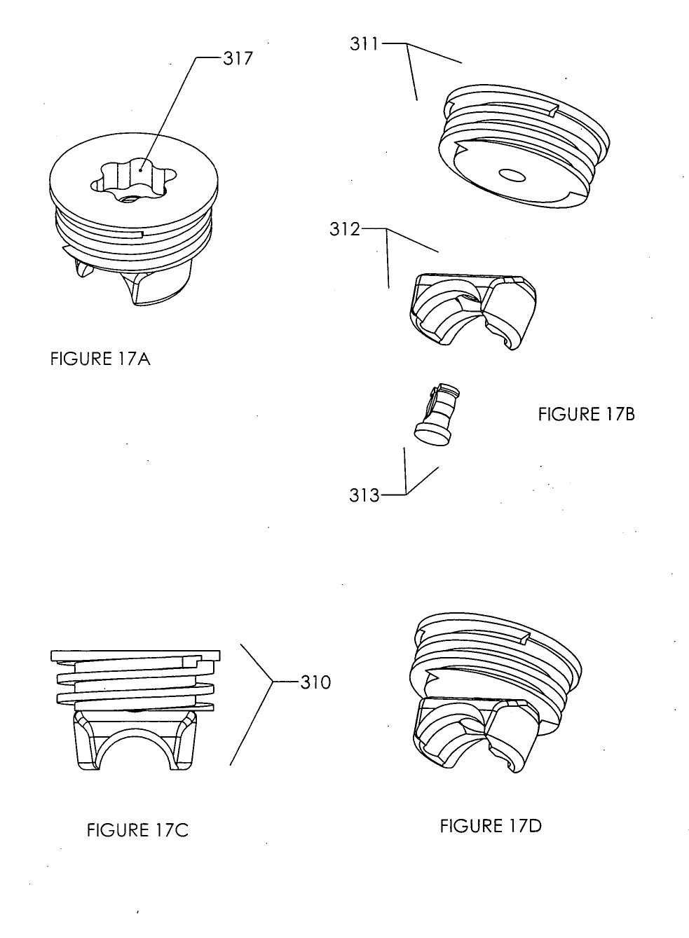

The locking cap assembly 310 illustrated in FIGS. 17A-D is a threaded embodiment. The assembly 310 comprises a drive member 311 (threaded in this embodiment) which when advanced drives the upper saddle 312 and lower saddle 330 together thereby restraining the outer member 121 while also driving the lower saddle 330 down to pinch and thereby lock the poly-axial bone screw 340 in a predetermined position with respect to the yoke 320. A restrainer 313 prevents separation of the lower saddle 330 from the drive member 311.

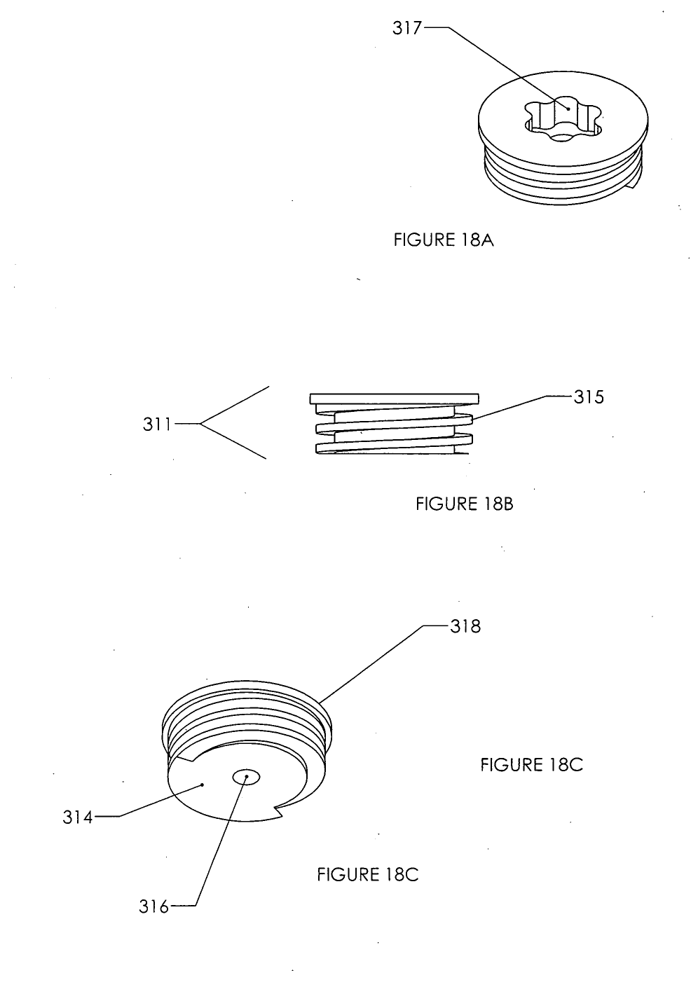

The drive member 311 further illustrated in FIG. 18A-C comprises a thread portion 315, a driving surface 314 for driving against the upper saddle 312, an aperture 316 for receiving the restrainer 313, and a drive recess 317 for advancing the drive member 311 utilizing an appropriate driver tool. The locking cap assembly 310 preferably includes a cap stop 318 shown here in the form of a rim on the cap to provide tactile feedback to the user to indicate the cap is fully advanced into the yoke 320.

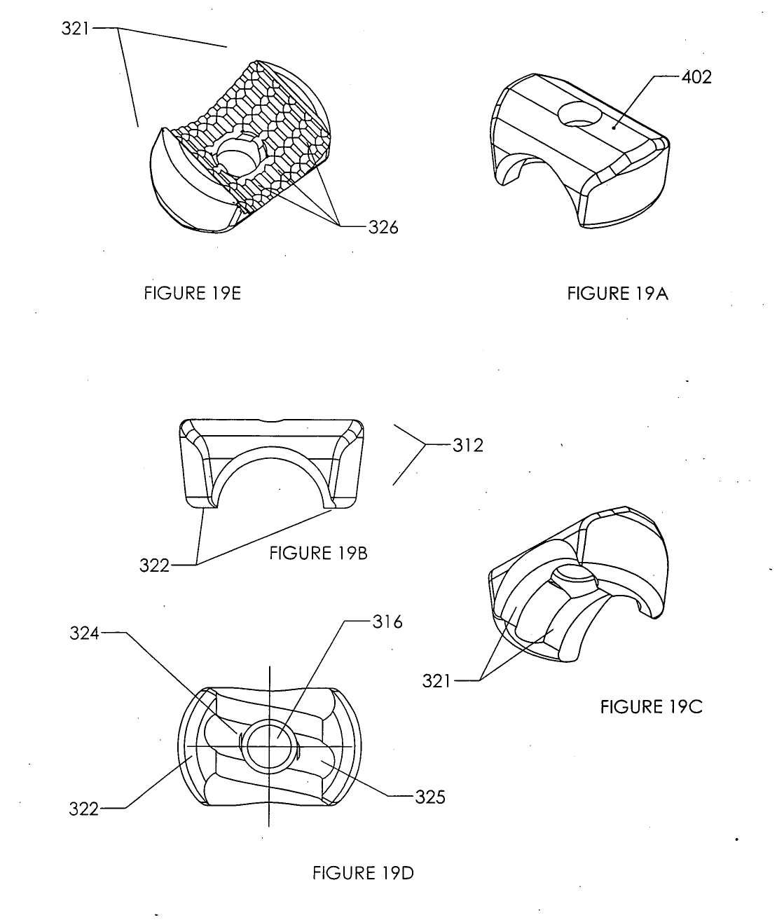

The upper saddle 312 of this embodiment is further illustrated in FIGS. 19 A-D. This component comprises an advancement face 402 driven down by the driving surface 314 when the drive member 311 is advanced. An aperture 316 is provided for receiving a portion of the restrainer 313 to keep the upper saddle 312 tethered to the drive member 311. The upper saddle 312 comprises a broad outer member restraint surface 321 intended to mate with outer surface portion 200 of the outer member 121 thereby preventing motion and accompanying wear from occurring therebetween. The perimeter of the saddle 312 is shaped to fit down the center of the yoke.

The upper saddle 312 further comprises saddle drive surfaces 322. These surfaces 322 will mate against opposing drive surfaces 322 on the lower saddle 330 to continue the transmission of compression forces when the drive member 311 is advanced to create screw 340 locking. These surfaces 322 also define the spacing between the upper saddle 312 and lower saddle 330 to create a predefined diameter outer member aperture 323 assuring the outer member 121 is restrained but doesn't overly compress against the inner member 130 causing undesired wear debris therebetween. Therefore, relatively even stress distribution about the outer member 121 is important for long term performance of this system 100. Pedicle screw designs which impart point contact on the outer sleeve are less desirable.

Again, the outer member restraint surface 321 is configured to mate with the outer surface portion 200 of the outer member 121 as described above. In this embodiment of FIG. 19D, the restraint surface 321 is configured with a helical groove 325 of geometry similar to the coiled outer member 121 illustrated in FIG. 3A. Further, coatings may be used between these surfaces to prevent undesired slippage therebetween. An anti-torsion element 324, preferably in the form of one or more ridges, grooves, or bosses may be mated with complementary elements on the outer surface of the coiled outer member 121 for torsion prevention. As yet another example illustrated in FIG. 19E, restraint surface 321 is configured with fixation elements 326 of predetermined dimension to carefully interlock with the ribs or grooves 210A and 210B of the outer member illustrated in FIGS. 14A & B.

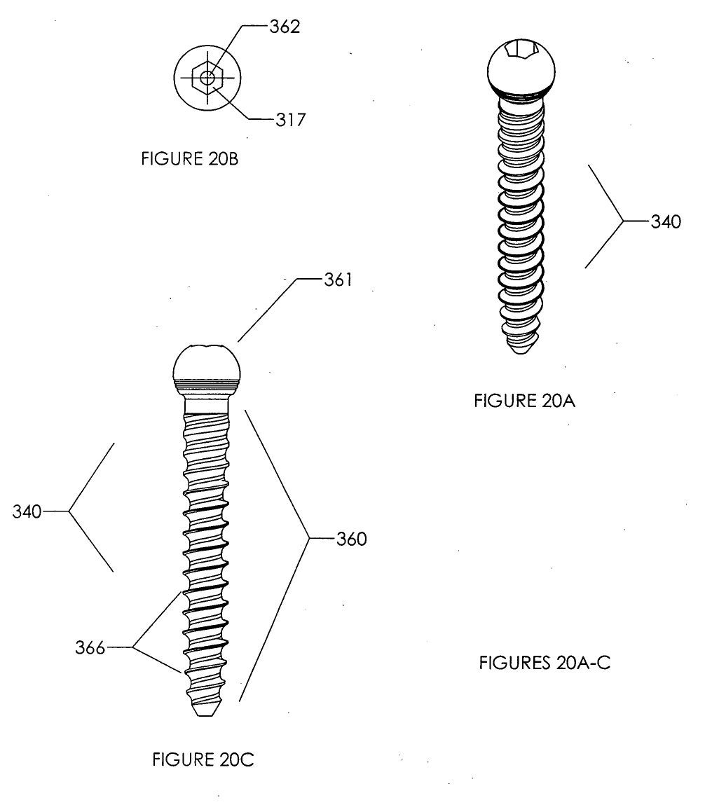

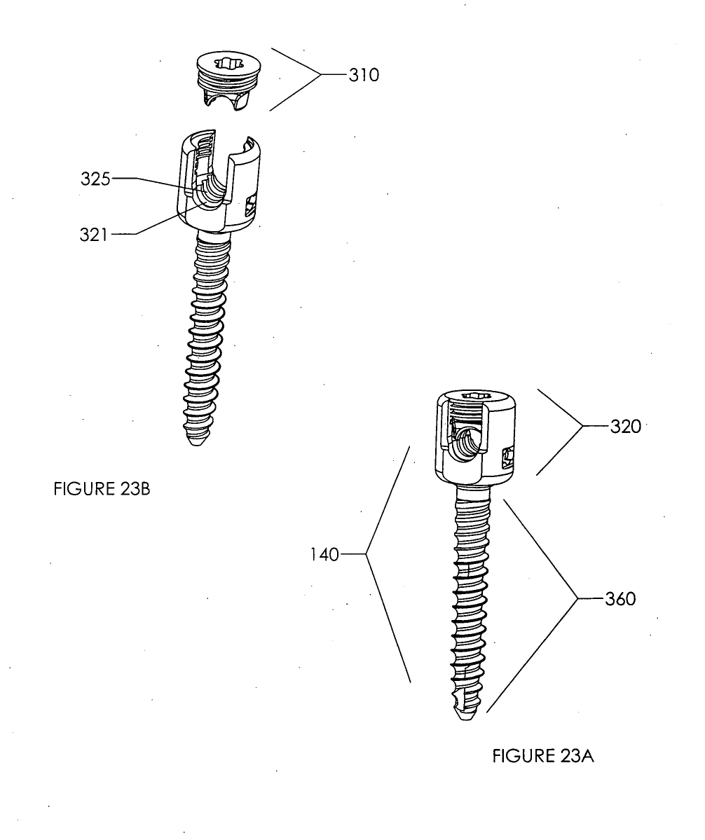

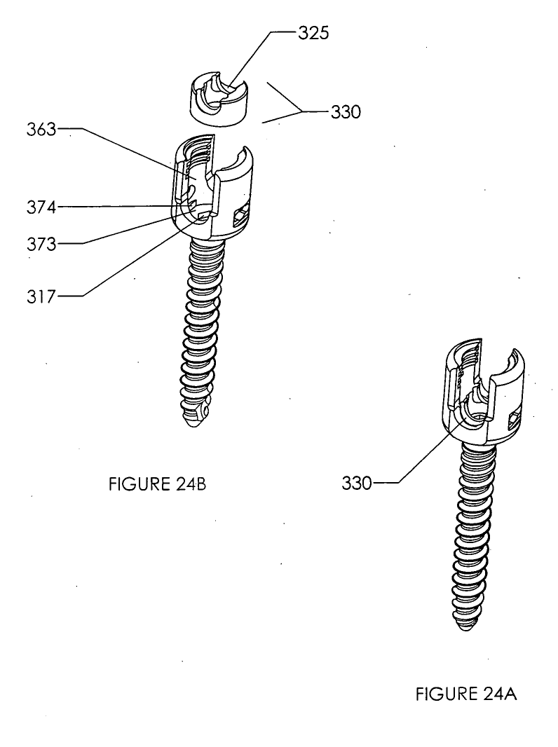

The bone screw 340 shown in FIG. 20 capable of poly-axial movement. This means that the shaft 360 of the screw 340 is capable of locking at multiple degrees of orientation with respect to the yoke 320. Bone screws that are non-polyaxial or fixed, most commonly have a shaft that is integral to the yoke 320 as illustrated in FIG. 23. The poly-axial bone screw 340 show in FIG. 20 comprises a spherical shaped head 361. The head 361 sits in the seat of the yoke 362 and its spherical shape assures that it will maintain continuous contact between the lower saddle 330 and the yoke 320 regardless of the angle of the screw. At the top of the head is a drive recess 317 for receiving a drive instrument for advancing the screw 340 into the vertebrae. The screw 340 may or may not have a cannula 362 per the preference of the surgeon. Such a cannula is generally used to advance the screw down a guidewire for minimally invasive placement. Bone screw threads 366 hold the screw in the vertebral body.

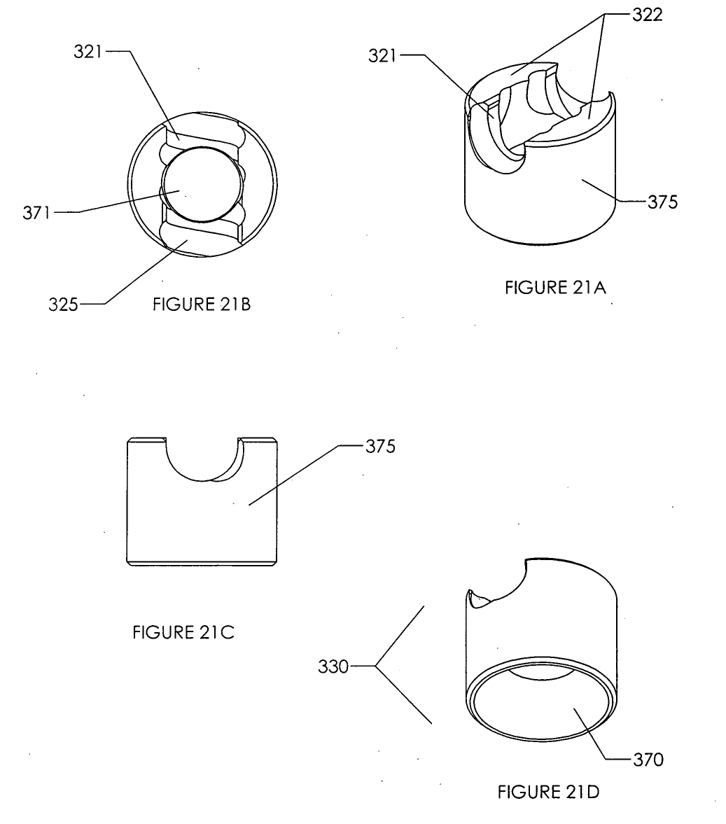

FIG. 21A-D illustrates a preferred embodiment of the lower saddle 330 for accommodating a poly-axial screw. This saddle 330 comprises a screw head recess 370 configured to mate with the screw head 361 primarily to transmit compression forces from advancing the drive member 311 therein locking the head 361 in a predetermined orientation with the yoke 320. The perimeter of the lower saddle 330 is sized to fit snug in the inner bore of the poly-axial yoke 320. The lower saddle 330 also comprises drive surfaces 322 to which mate with those on the upper saddle for the functions explained previously. Similar to the upper saddle, an outer member restraint surface 321 is configured to mate with the outer surface portion 200 of the outer member 121. In this embodiment it is configured with a helical groove 325 to carry the spring coils 123 of the outer member in FIG. 3A, but as discussed earlier, it is best configured to cooperate with the outer member restraint surface 321. A central aperture 371 provides access for instruments to advance the bone screw 140.

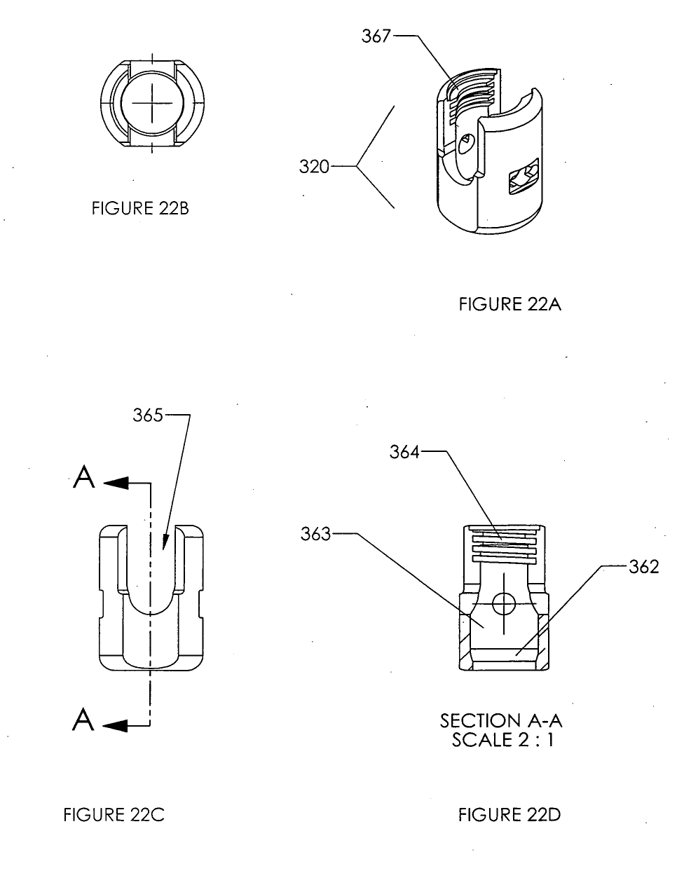

The yoke 320 is utilized to hold the primary components of the spinal stabilization system 100 together. Illustrated in FIG. 22A-D is an example of one embodiment of a yoke 320 suited for a poly-axial screw 340 as described in 20A and a threaded style locking cap assembly 310 as described in 17A. The poly-axial style yoke comprises a seat 362 for seating of the screw head 361, an inner chamber 363 for the head 361 to reside, internal or external threads or grooves 364 for advancement of the locking cap assembly 310, and an elongate member canal 365 configured to receive the elongated stabilization member 120. Yoke stop 367 interferes with cap stop 318 when the drive member 311 is fully deployed to the predetermined position.

FIG. 23A and FIG. 23B illustrate an example of a threaded fixed pedicle screw 140 configured for this spinal stabilization system 100. Fixed screws are known to be more reliable than poly-axial screws since the shaft 360 is typically machined integral to the yoke 320 eliminating any chance for slippage between the yoke 320 and screw head 361. This embodiment utilizes the same locking cap assembly 310 illustrated previously in FIG. 17A. A differentiator for this embodiment is the outer member restraint surface 321 is machined integral to the floor of the yoke 320 with a helical groove 325 of geometry similar to the coiled outer member 121 illustrated in FIG. 3A. This integral restraint surface 321 eliminates the need for a lower saddle 330. However, manufacturing difficulties may warrant a fixed screw having a separate lower saddle 330 as illustrated in FIGS. 24A and 24B.

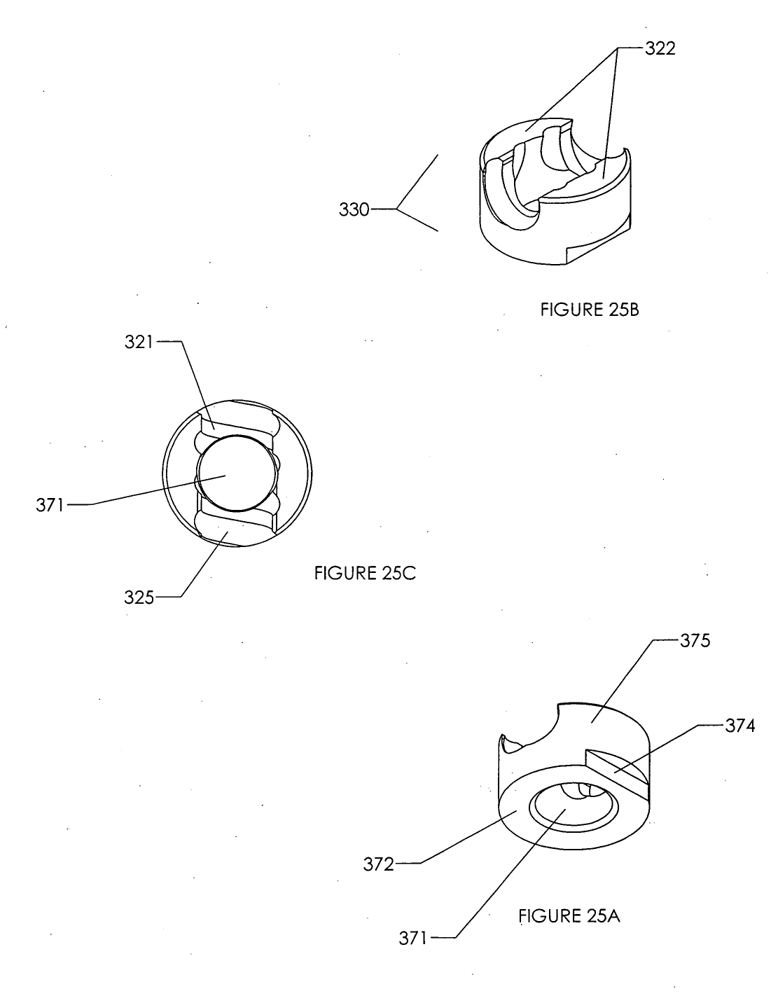

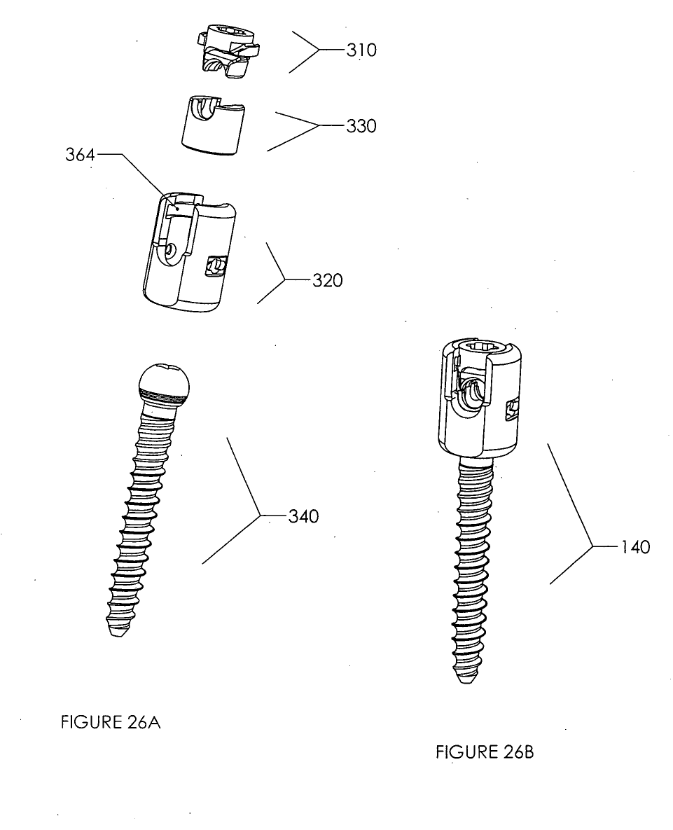

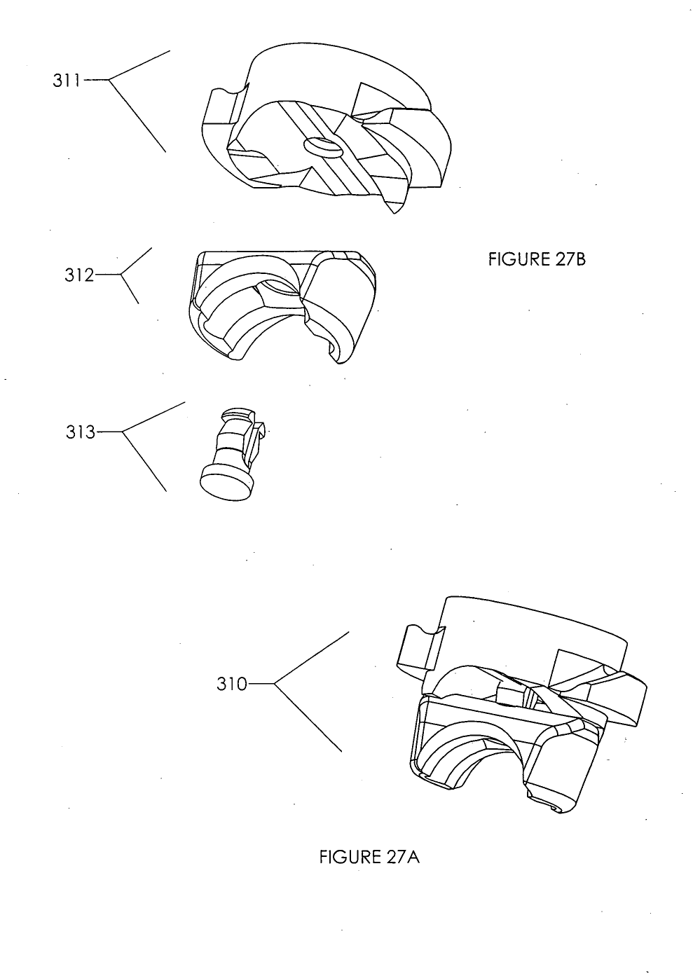

The lower saddle 330 in 24A is further illustrated in FIGS. 25A-C. This lower saddle 330 shares many of the same features of the saddle illustrated in FIGS. 21A-D. However, the saddle 330 in FIGS. 25A-C is configured for a fixed screw wherein the shaft 360 is integrated to the yoke 320. There is no screw head 361 for the yoke 320 to seat, therefore this saddle 330 is absent a screw head recess 370. The saddle base 372 in FIG. 25A rests on the floor 373 of the inner chamber 363. The saddle base 372 or perimeter wall 375 of the FIGS. 21 and 25 may further comprise an anti-torsion element 374 in the form of a notch, ridge, boss, recess or other form to cooperate with a complementary element 374 on the floor 373 or inner chamber 363 side wall to prevent the saddle 330 from unintentionally falling out of the yoke 320 and for prevention of rotation between lower saddle 330 and yoke 320. The yoke 320 of the fixed variety also comprises a drive recess 317 to drive the implant into bone.

As an alternative embodiment to pedicle screws 140 described above, a poly-axial screw 140 with locking cap assembly 310 of the flanged variety may be implemented as illustrated in FIGS. 26A-B, 27A-B, and 28A-D. The upper saddle 312 and restrainer 313 in this embodiment mirror those described earlier. The drive member 311 comprises one or more flanges 400 that is substantially flattened and configured to reside in the groove 364 formed in the yoke 320 wall. The driving surfaces 314 formed on the bottom side of the drive member 311 are sloped and cooperate with the advancement face 402 on upper saddle 312 to advance saddle 312 toward the outer member 121 therein locking the construct. Alternatively, the flanges 400 could be inclined, much like a single thread, provided the groove 364 formed in the yoke 320 is correspondingly inclined. In such a configuration, inclined driving surfaces 314 that are sloped may be unnecessary.

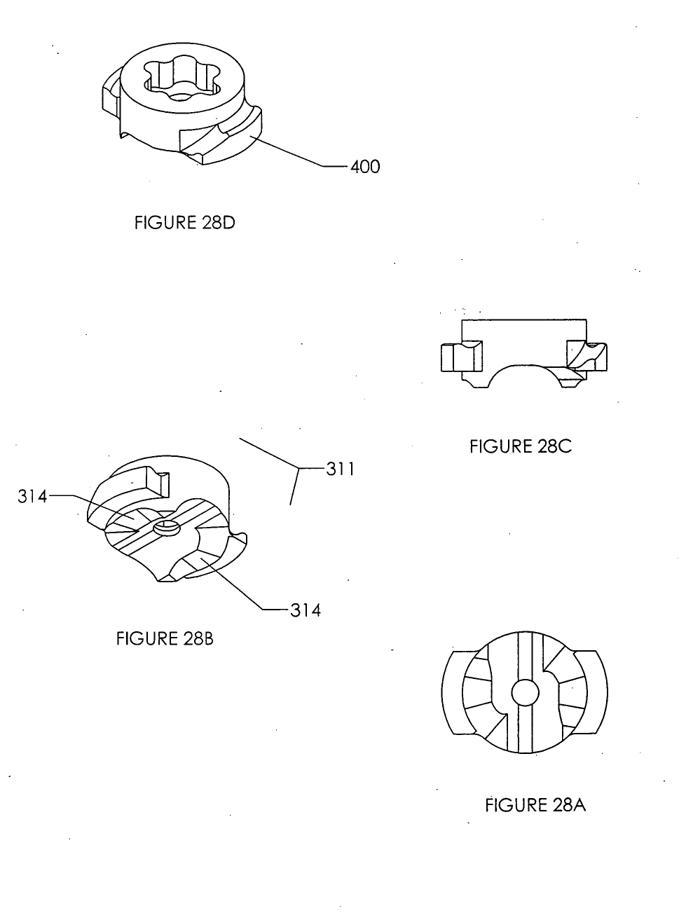

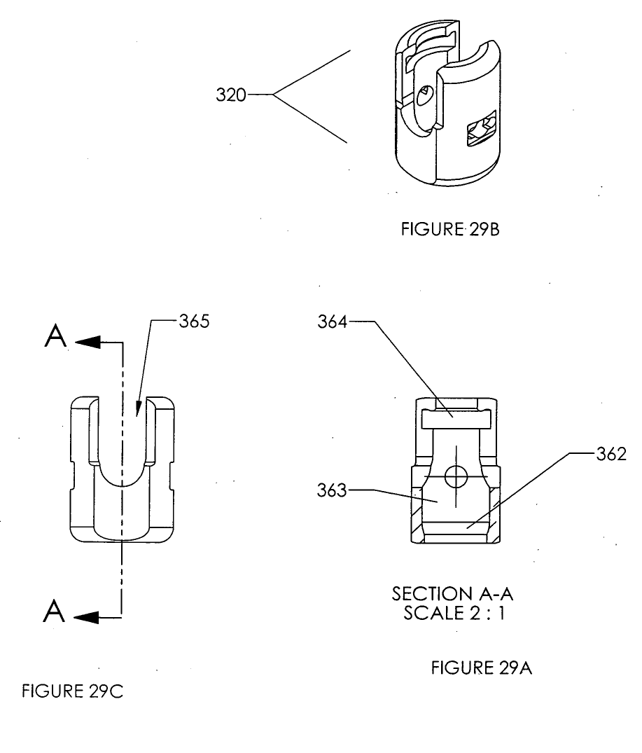

A yoke 320 of the poly-axial variety, configured to operate with the cap described in FIGS. 28A-D is illustrated in FIGS. 29A-C. This yoke 320 shares common features of the yoke illustrated in FIG. 22A-D with the exception that the recess in the wall is a groove 364 as opposed to a thread. The screw and thread arrangement could be reversed such that the groove resides on the cap and the flange resides on the yoke.

The pedicle screws 140 described here are only a few examples of screws 140 that could be utilized with this stabilization system 100. Clearly, pedicle screws of other varieties such as those that are side loading, lock through sliding an inner member over an outer member, utilize snap in caps, have caps engaging the outside of the yoke, and other functional designs, could easily implement similar features described herein to cooperate with specialized elongated stabilization member 120 to produce similar results.

Although the apparatus disclosed herein has been described with respect to preferred embodiments, it is apparent that modifications and changes can be made thereto without departing from the spirit and scope of the invention as defined by the claims.

1. A spinal implant system for stabilization of the spine comprising: a pair of bone anchors; an elongate stabilization device received in the bone anchors, the stabilization device having an elongate inner stabilizing member and an outer stabilizing member disposed about the inner member; wherein said anchors are configured to inhibit translation of the outer member and to permit translation of the inner member.

2. The system of claim 1 wherein said outer member is configured to bias the first and second bone anchors towards each other at a predetermined point during flexion movements of the spine and away from each other at a predetermined point during extension movement of the spine.

3. The system of claim 1, wherein the outer stabilizing member is an elastically deformable spring that compresses during spinal extension to provide a gradual increase in resistance to further spinal extension.

4. The system of claim 3 wherein when the outer member is fully compressed further spinal extension movement is completely inhibited.

5. The system of claim 1 wherein the anchor members each comprise a stabilization member housing with grooves configured to receive portions of the outer stabilizing member and inhibit translation of the outer stabilizing member with respect to the anchor housing members without exerting compression on the inner elongated member.

6. The system of claim 5 wherein the bone anchors each further comprise a locking cap with a lower surface having recesses configured to receive portions of the outer stabilizing member and inhibit translation of the outer stabilizing member with respect to the anchor housing members without exerting compression on the inner elongated member.

7. The system of claim 1 wherein at least one bone anchor further comprises a locking cap with a lower surface configured to receive portions of the outer stabilizing member and inhibit translation of the outer stabilizing member with respect to one anchor housing member without exerting compression on the inner elongated member.

8. A spinal implant system for stabilization of the spine comprising:
a first bone anchor coupled to a first housing with a channel therethrough open at the top and two sides for receiving an elongate stabilization device and a first lock member for closing the channel from the top;
a second bone anchor having a head portion pivotably received in a second housing portion, the housing portion having a channel therethrough open at the top and two sides for receiving an elongate stabilization device and a second lock member for closing the channel from the top;
an elongate stabilization device received in the bone anchors, the stabilization device having an elongate inner stabilizing member and an outer stabilizing member disposed about the inner member;
wherein the first and second housings and the first and second lock members cooperate to inhibit translation of the outer member and to permit translation of the inner member;
wherein the second lock member has arms that extend over the stabilization device to exert a compressive force on the head of the second bone anchor to inhibit pivoting with respect to the second housing without exerting a clamping force upon the stabilization device.

9. The system of claim 8 wherein a compression member is disposed within the second housing and in contact with the head portion of the second bone anchor, and the arms of the second lock member engage the compression member to force the compression member against the head portion to prevent the head portion from pivoting with respect to the second housing.

10. The system of claim 9 wherein the second locking cap comprises an upper saddle member and the compression member comprises a lower saddle member, wherein the upper saddle member is configured to directly compress the lower saddle member to positionally fixing the pedicle screw to yoke angle without compressing agains the stabilization device.

11. A pedicle screw comprising a shank portion and a coupling portion, the coupling portion having a channel for receiving an elongate stabilization member, the channel forming an inner surface of the coupling portion; wherein a portion of the inner surface is configured to restrain at least a portion of an elongate stabilization member against translation without compression of the stabilization member.

12. The screw of claim 11 wherein the shank portion is integral to the coupling portion.

13. The screw of claim 11 wherein one end of the shank portion is pivotably received within the coupling portion.

14. The screw of claim 11 further comprising a lower saddle and an upper saddle configured to capture the elongate stabilization member without compression of the elongate stabilization member.

15. The screw of claim 14 wherein the shank portion is integral to the coupling portion and the upper saddle forms a lower portion of a locking cap that is rotatably secured to the coupling portion.

16. The screw of claim 15 wherein the stabilization member comprises a helical outer member, and wherein the upper saddle and lower saddle have grooves for receiving the outer member and preventing translation of the outer member with respect to the coupling portion.

17. The screw of claim 14 wherein one end of the shank portion is pivotably received within the coupling portion and the upper saddle forms a lower portion of a locking cap that is rotatably secured to the coupling portion.

18. The screw of claim 17 wherein the stabilization member comprises a helical outer member, and wherein the upper saddle and lower saddle have grooves for receiving the outer member and preventing translation of the outer member with respect to the coupling portion.
